(19) United States
(12) Patent Application Publication
(10) Pub. No.: US 2005/0203465 A1
(43) Pub. Date: Sep. 15, 2005

(54) Needle for combined epidural intradural anesthesia

(76) Inventor: Edmundo-Angel Mateo Llurba, Monteagudo (ES)

Correspondence Address: JACOBSON HOLMAN, 400 SEVENTH STREET, N. W., WASHINGTON, DC, 20004, US

(21) Appl. No.: 11/058,428

(22) Filed: Feb. 16, 2005

(30) Foreign Application Priority Data
Feb. 27, 2004 (ES) 2004.00 340/5

(51) Int. Cl. A61M 5/00

(52) U.S. Cl. 604/239

(57) ABSTRACT

The present invention discloses an improved needle for combined epidural and intradural anesthesia comprising two rigid elongated tubular members joined lengthways for guiding an epidural catheter and an intradural needle, respectively, wherein the tubular members are linked in their rear parts to a common nucleus, which externally defines an anatomic shape adapted for handling it with fingers and internally includes respective canals that extend from the rear parts to connect with the tubular members. The canals have conic shapes and their openings at rear part have shapes for linkage of conventional syringes for anesthetic application.

### FIELD OF THE INVENTION

This invention is related to local anesthesia applied in the epidural area and specifically to the one applied with combined techniques using intradural anesthesia, including a needle especially for facilitating the application of both types of anesthesia through the same puncture element but separate canals.

### DESCRIPTION OF PRIOR ART

The application of the epidural anesthesia technique is increasing in cases such as deliveries in which an effective partial anesthesia is required and when it may be necessary to extend the anesthesia for an unspecified period of time. A puncture is applied for these purposes by using a special needle to the epidural area. This needle will be removed after introducing a catheter through it. The catheter is provisionally installed as the canal for supplying the necessary intradural anesthetic for maintaining an appropriate postoperative analgesia.

This type of anesthesia proves to be slow in regard to the commencement of its effect. Therefore, a combined application technique with intradural anesthesia has been used lately. The effect of such technique is much faster. A short application of intradural anesthesia results in the commencement of the effect of the local anesthesia and then the effect can be extended as necessary through epidural anesthesia.

Currently, the application of this type of anesthesia (combined) is carried out either by using the epidural needle as an introducer or guide for the intradural needle and placing the catheter once this is performed, or, by puncturing in separate areas. This entails difficult and slow procedures and applications both for placing the catheter after the intradural is performed and for losses (in many cases) of spatial reference of location and anesthetic depot. There are certain needles with a short parallel guide, but with some structural characteristics that make it very difficult to prepare and carry out the technique.

### SUMMARY OF THE INVENTION

In accordance with the invention, a needle for the combined application of the two types of anesthetics mentioned above is suggested. This needle has been designed according to the constructive and functional characteristics for a favorable application.

This needle, which is the object of the invention, consists of two rigid tubular metallic elements, of different diameters, which are joined lengthways parallel to each other. Such elements are connected to the rear part of a nucleus of synthetic material, such as polycarbonate or other similar materials. This nucleus determines an external anatomic shape which facilitates handling and gives shape to two entries with linkages for conventional type syringes and for the intradural needle. From these entries, respective conic canals are internally defined until the connection of the tubular metallic elements.

An easy-to-handle needle for anesthetic puncture is then obtained, which allows the easy introduction of the epidural catheter and the intradural needle through it at the same time, since the conic canals of the nucleus facilitate the insertion through the tubular metallic elements, without any decantation problems, while the nucleus itself facilitates attachment of the set when the operation is performed.

In addition, in relation to the tubular metallic elements, respective fasteners are incorporated for a provisional closure during puncture to prevent such elements from being blocked and causing possible problems for later anesthetic applications.

Therefore, functional advantages are obtained with the recommended needle in the aspect of facilitating focus, guidance and exact positioning of the intradural needle in the dura. The needle also allows for the insertion of the epidural catheter before the intradural puncture. In addition, only one puncture is necessary to obtain a double canal for introducing the epidural catheter and the intradural needle separately. The simple and comfortable handling has an impact on the patient and the doctor performing the operation to avoid accidental insertions of the catheter through the dura from the orifice of the intradural puncture at the same time.

Therefore, such a needle, which is the subject matter of this invention, certainly proves to have very advantageous characteristics. It has the preferable characteristics for the application in the combined anesthesia operation.

### BRIEF DESCRIPTION OF THE DRAWINGS

FIG. 1 shows a front view of a sample needle according to the invention.

FIG. 2 shows the corresponding lateral side view with respect to the previous figure.

FIG. 3 shows the top plan view when the needle is seen from above.

FIG. 4 shows an enlarged partial sectional view of the needle with a partial cut of the nucleus.

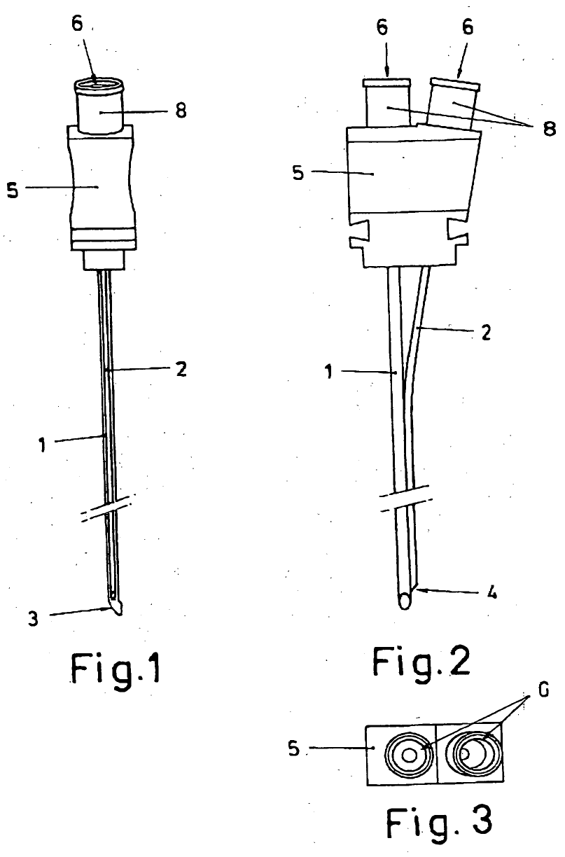

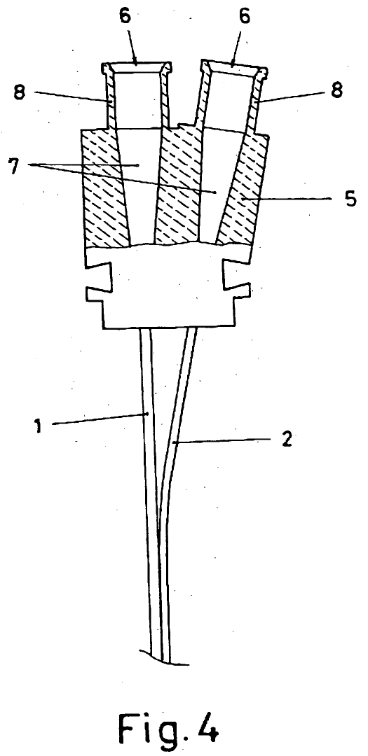

### DETAILED DESCRIPTION OF THE INVENTION

The object of the invention refers to a needle used in the application of combined epidural and intradural anesthesia, with constructive characteristics that facilitate the operation to be performed.

The recommended needle consists of two metallic tubular members (1 and 2) of rigid material, which are joined lengthways parallel to each other, for example through laser welding. One member (1) is of a larger diameter than the other member (2).

The member (1) with a larger diameter (thicker member) has a length that exceeds the length of the other member (2) (thinner member) at the front section, wherein the front section of such thicker member (1) has a curved end (3), which leads the opening to an opposite direction of the position in which the thinner member (2) is placed, which has an oblique cut at its end (4).

The two tubular members set (1 and 2) is linked to a nucleus in the rear part (5), which has an external anatomic shape that facilitates its handling with fingers when the needle is used during operations.

In the rear part of the nucleus (5), there are two openings (6), which extend internally according to their respective canals (7) of conic shape, which will end respectively in correspondence with the tubular elements (1 and 2), determining a decantation in relation with themselves.

Through the external part, the openings (6) define certain shapes (8) with respect to which the conventional syringes used in anesthetic applications, such as the “luer record” and “luer lock” types, are capable of linkage.

The device with structures as described above allows the introduction of an epidural catheter through the tubular member (1) and the introduction of an intradural needle through the tubular member (2), for the combined application of these two types of anesthesia, so that both applications can be carried out through the same puncture element but through different guide canals, allowing the nucleus (5) to maintain the set perfectly attached during the operation.

The conic canals (7) of decantation towards the tubular members (1 and 2) facilitate the introduction of the elements to be inserted and a fast and safe operation. At the same time, the arrangement of the exit ends of the tubular members (1 and 2) allows for a precise determination of the puncture with the intradural needle through the dura, from the tubular member (2). While the exit direction of the tubular member (1) completely avoids the risk of the epidural catheter being inserted using the puncture of the intradural needle and through the dura when introduced.

The tubular members (1 and 2), which are the ones used for performing the puncture through to the epidural area, can be provided with reference marks in order to control the insert measure during puncture and facilitate the insertion until the precise point is reached without mistakes.

On the other hand, in relation to the interior of the aforementioned tubular members (1 and 2), respective closure fasteners can be provisionally included in order to avoid corporal matter from entering into such elements during puncture and causing problems for the later introduction of the catheter and the needle for anesthetic applications.

1- An improved needle for combined epidural and intradural anesthesia, said needle comprises two rigid elongated tubular members joined lengthways and forming a puncture set for guiding an epidural catheter and an intradural needle, respectively, wherein the tubular members are joined at their rear parts to a common nucleus, which externally defines an anatomic shape adapted to be handled with fingers and internally includes respective canals which extend from the rear parts to connect with the tubular members.

2- The improved needle for combined epidural and intradural anesthesia, in accordance with claim 1, wherein the respective canals are of conic shape and each join the respective tubular members.

3- The improved needle for combined epidural and intradural anesthesia in accordance with claim 1, wherein, the respective canals in the nucleus have ends opposite the tubular members with a shape for connecting to conventional syringes for anesthetic application.
